(19) United States
(12) Patent Application Publication
(10) Pub. No.: US 2022/0257962 A1
(43) Pub. Date: Aug. 18, 2022

(54) BODY SUPPORT SYSTEM FOR EMITTING ULTRA-LOW RADIO FREQUENCY ENERGY AND ASSOCIATED SYSTEMS AND METHODS

(71) Applicant: Hapbee Technologies, Inc., Vancouver (CA)

(72) Inventors: Brian Mogen, San Francisco, CA (US); Gerard P. Murray, Carlsbad, CA (US); Scott Donnell, Phoenix, AZ (US)

(21) Appl. No.: 17/671,326

(22) Filed: Feb. 14, 2022

Related U.S. Application Data
(60) Provisional application No. 63/149,137, filed on Feb. 12, 2021.

(51) Int. Cl. A61N 1/40; A61M 21/02

(52) U.S. Cl. CPC A61N 1/40; A61M 2021/0055; A61M 21/02

(57) ABSTRACT

The present technology is generally directed to energy-emitting body support systems for emittingultra-low radiofrequency energy (“ULRE”), and associated devices and methods. In some embodiments, an energy-emitting body support system includes an emission or coil assembly having one or more emission elements or coils. Each of the emission elements can be configured to emit one or more ULRE signals to apply one or more stimulation regiments to a user. At least some of the stimulation regiments are expected to cause the user to achieve a predefined state (e.g., calm, tired, restful, energized, motivated) when applied to the user. The body support system can further include an emission assembly substrate carrying one or more of the emission elements and configured to support at least a portion of the user in a recumbent or seated position during delivery of the stimulation regiments.

### CROSS-REFERENCE TO RELATED APPLICATION

This application claims priority to U.S. Provisional Pat. No. 63/149,137, titled “BODY SUPPORT SYSTEM FOR EMITTING ULTRA-LOW RADIO FREQUENCY ENERGY,” filed Feb. 12, 2021, the entirety of which is incorporated by reference herein.

### TECHNICAL FIELD

The present technology is directed to devices, systems, and methods for supporting a human body or portions thereof in a recumbent or seated position and emitting ultra-low radio frequency energy.

### BACKGROUND

Body supports, such as mattress, mattress pads, mats, cushions, and the like, are commonly used to improve a user's comfort in a variety of positions or postures. However, while some body supports may be able to improve a user's physical comfort, many body support devices lack the ability to actively affect a user's mental or emotional state to attain a desired state of wellbeing.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1 is an exploded isometric view of an energy-emitting body support system configured in accordance with embodiments of the present technology.

FIG. 2 is a partially schematic illustration of an energy-emitting body support system and associated environment configured in accordance with some embodiments of the present technology.

FIGS. 3-5 are isometric views of an emissions assembly in a flat state, a partially folded state, and a folded state, respectively, in accordance with embodiments of the present technology.

### DETAILED DESCRIPTION

The present technology is generally directed to body support systems for emitting ultra-low radiofrequency energy (“ULRE”), and associated devices and methods. In some embodiments, an energy-emitting body support system includes a support substrate and an emissions assembly integrated in or supported by the support substrate. The emission assembly can include one or more emission components (also referred to as “emission elements”), such as coils, configured to emit one or more ULRE signals, e.g., to create a magnetic field and/or apply one or more stimulation regiments (e.g., electrical stimulation) to a user. Each of the ULRE signals can include one or more predefined signal delivery parameters (e.g., frequency, amplitude, bandwidth, duration, active/inactive emission element(s), etc.). When the user is positioned on or near the body support system and the emissions assembly produces ULRE signals in accordance with one or more of the predefined signal delivery parameters and/or a stimulation regiment, the ULRE signals are expected to cause the user to achieve a predefined mental or physical state or sensation (e.g., calm, tired, restful, energized, motivated, focused, pain relief, alert, tired, sleepy, and the like) associated with the predefined signal delivery parameters and/or the stimulation regiment. In at least some embodiments, for example, the body support system includes a mattress pad carrying an emission assembly with a plurality of emission elements, and individual ones of the emission elements are configured to deliver one or more stimulation regiments that are expected to produce a calming sensation, e.g., to help a user fall asleep faster and/or improve the user's sleep quality.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section.

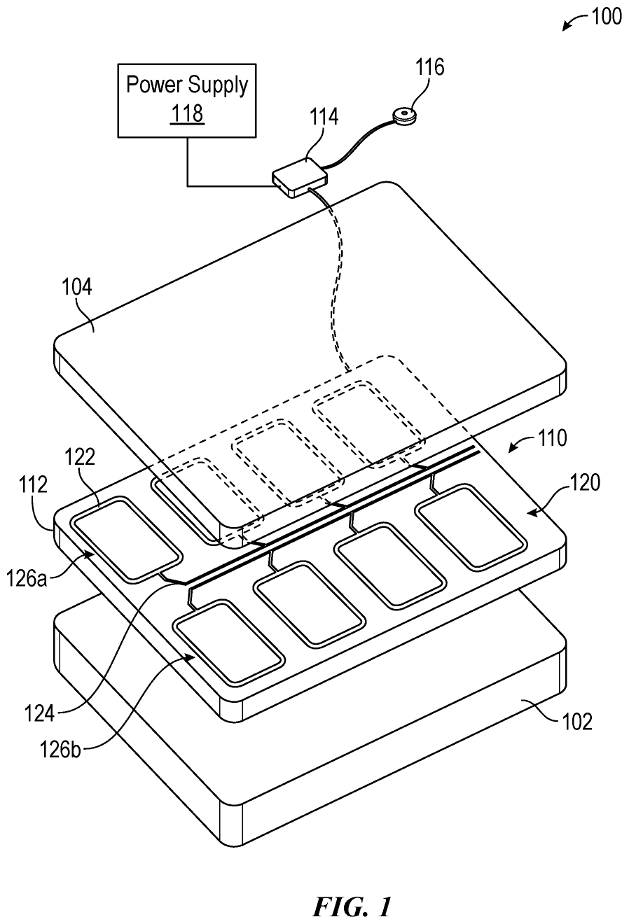

Specific details of several embodiments of the technology are described below with reference to FIGS. 1-5. Although many of the embodiments are described below with respect to devices, systems, and methods associated with mattresses, other applications and other embodiments in addition to those described herein are within the scope of the technology. For example, the present technology may be used with other structures that can support a humanbody, such as mattress toppers, pillows, neck pillows, pads (e.g., mattress pads, camping pads), mats (e.g., yoga mats), couches, recliners, chairs, office chairs, massage chairs, wheelchairs, car seats, car seat covers, massage tables, and other supporting devices and/or support systems. Additionally, several other embodiments of the technology can have different configurations, components, and/or procedures than those described herein, and features of the embodiments shown can be combined with one another. A person of ordinary skill in the art, therefore, will accordingly understand that the technology can have other embodiments with additional elements, or the technology can have other embodiments without several of the features shown and described below with reference to FIGS. 1-5.

Reference throughout this specification to “one embodiment” or “an embodiment” means that a particular feature, structure, or characteristic described in connection with the embodiment is included in at least one embodiment of the present technology. Thus, the appearances of the phrases “in one embodiment,” “in an embodiment,” and the like in various places throughout this specification are not necessarily all referring to the same embodiment. Furthermore, the particular features or characteristics may be combined in any suitable manner in one or more embodiments. Reference throughout this specification to relative terms such as, for example, “generally,” “approximately,” “substantially,” and “about” are used herein to mean the stated value plus or minus 10%.

### Selected Embodiments of Energy-Emitting Body Support Systems

FIG. 1 is a partially exploded isometric view of an energy-emitting body support system 100 (“body support system 100”) configured in accordance with embodiments of the present technology. The body support system 100 can include an emission assembly 110 (which can also be referred to as a “signal delivery assembly,” a “stimulus assembly,” a “coil assembly,” and the like) that can be configured to emit one or more wireless signals (e.g., ULRE) to apply stimulation to and/or otherwise produce an effect in a user positioned on or proximate to the emission assembly 110 (i.e., within the emissions zone). The emission assembly 110 can have a stimulation transmission distance of between about 0.01 mm to about 50 cm, such as at least 1 cm, 10 cm, 20 cm, 30 cm, any number therebetween, or any other suitable stimulation transmission distances. The stimulation transmission distance of the emission assembly 110 can define, at least in part, an emissions zone in which the emission assembly 110 can impart a predefined effect on a user when the user is posited within this emissions zone. As shown in the illustrated embodiment, the body support system 100 can further include an emission assembly substrate 112 (“substrate 112”) that carries the emission assembly 110, at least one controller 114 operably coupled to the emission assembly 110, and at least one control station 116 operably coupled to the emission assembly 110. The controller 114 can control the overall the operation of the emission assembly 110 (e.g., power, activation, signal strength and type, signal emissions regime), and the control station 116 can be accessible by the user (e.g., positioned on a nightstand) to provide a user interface for activating and/or otherwise controlling the emission assembly 110.

As shown in FIG. 1, the emission assembly 110 and the substrate 112 can be positioned between a first substrate 102 and a second substrate 104. In the illustrated embodiment, the first substrate 102 is a mattress, the second substrate 104 is a fitted mattress pad or sheet, and the emission assembly 110 is positioned therebetween in a similar manner as a mattress pad to keep the emission assembly 110 securely positioned between first substrate 102 and the second substrate 104. The mattress and mattress cover can be any mattress size (e.g., twin, double, full, queen, king California king, etc.). In these embodiments, the emission assembly 110 can be configured to apply stimulation to a user on (e.g., lying on) the mattress (e.g., before, during, and/or after sleep). In various embodiments, the emission assembly 110 can be coupled to or integrated with the mattress itself, the fitted sheet 104, and/or other type of padding or structure that is positioned under a person when lying down. In these and other embodiments, the first support substrate 102 and/or the second support substrate 104 can each have any other suitable configuration and/or be omitted.

The substrate 112 may be made from one or more materials that are suitable fora person to lay on, sit on, and/or otherwise support body weight. In some embodiments, for example, the substrate 112 can be made from a pliable and/or a soft material, such as foam or other padding (e.g, including one or more of rubber, plastic, cork, hessian, vinyl, jute, cotton), a fabric (e.g., cotton, vinyl cotton, polyester), and/or a fill material (e.g., polyester fiberfill, wool, cotton, down, polyurethane). In these and other embodiments, the substrate 112 can include semirigid materials, rigid materials, and/or any other suitable material or combination of materials. In some embodiments, the substrate 112 may be suitable for placing on top of a person, similar to a blanket.

The emission assembly 110 includes at least one emission element array 120, and each emission element array 120 can include one or more emission elements 122. Individual ones of the emission elements 122 can include one or more coils of wire (e.g., copper wire), one or more flexible conductive printed circuits, and/or any other suitable emission elements that can emit ULRE. In at least some embodiments, one or more of the emission elements 122 include a Loomia Electronic Layer (LEL) or other suitable emission element manufactured by Loomia Technologies, Inc., headquartered in New York, N.Y. Individual ones of the emission elements 122 can be flat, circular, rectangular, and/or have any other suitable shape. The emission elements 122 can be carried by, embedded in, and/or otherwise coupled to the substrate 112. For example, one or more of the emission elements 122 can be coupled to the substrate 112 via an adhesive, interfacing grooves, a coupling mechanism, and/or any other suitable coupling technique configured to reduce or prevent movement of the emission element 122 relative to the substrate 112.

In the illustrated embodiment, the emission element array 120 includes eight emission elements 122 arranged in a two-by-four grid pattern. In other embodiments, the emission element array 120 can include more or fewer emission elements, such as at least one, less than eight, more than eight, or any other suitable number of emission elements. Additionally, or alternatively, the emission elements 122 can be arranged in any other suitable pattern, including one or more rows and/or columns having any suitable number of emission elements, a zig-zag pattern, a circular pattern, a triangular pattern, a square pattern, a rectangular pattern, or any other suitable pattern. In some embodiments, the emission elements 122 can be distributed/spaced evenly across the emission element array 120. In further embodiments, the emission elements 122 can be clustered/grouped in a single region of the emission element array 120. In these and other embodiments, at least some of the emission elements 122 can be positioned to be at least partially aligned with and/or stimulate one or more regions of a user's anatomy (e.g., upper body, lower body, lower back, foot/feet, arm/arms, head, neck, etc.).

The emission element array 120 can deliver stimulation to one or more users positioned on or proximate to the emission element array 120 (i.e., within the stimulation transmission distance). In the illustrated embodiment, for example, the emission element array 120 includes a first row 126a of emission elements 122 positioned to be at least partially aligned with a first user and a second row 126b of emission elements 122 positioned to be at least partially aligned with a second user, such that the first and second users can receive a same or different stimulation from the respective rows 126a, 126b of the emission element array 120. Additionally, although described with reference to two users, it will be appreciated that the first and second rows 126a, 126b can be aligned with a single user, e.g, the first row 126a with a first (e.g., right) side of the single user and the second row 126b with a second (e.g., left) side of the single user opposite the first side.

Each of the emission elements 122 can be individually activated and configured to emit one or more radiofrequency (“RF”) energy signals, e.g., to create a magnetic field and/or apply one or more stimulation regiments (e.g., electrical stimulation) to a user. In at least some embodiments, the RF energy signals can include ultra-low RF energy (“ULRE”) signals. The stimulation signals (also referred to as a “stimulation regiment” or a “cognate”) can include one or more specific time-series waveforms or signal delivery parameters (e.g., frequency, amplitude, bandwidth, duration, active/inactive emission element(s), etc.). In some embodiments, the signal delivery parameters include a frequency of between about 0 kHz to about 50 kHz, a bandwidth of about 50 kHz, and/or a magnetic field amplitude (e.g., a root mean squared amplitude) of up to about 50 mGauss. In other embodiments, the stimulation regiments can have any other suitable signal delivery parameters and/or signal delivery parameter values.

The stimulation signals and/or the signal delivery parameter values thereof are expected to replicate one or more effects of specific substances. These substances can include: melatonin, adenosine, CBD, THC, caffeine, nicotine, adenosine, theobromine, alcohol, beta-endorphin, methysticin, osthole, isoamyl acetate, kavain, 2-methyl-4-vinylphenol, leptin, oxytocin, dextromethorphan, ghrelin, 1-theanine, bacopa extract, 5-hydroxy-L-tryptophan (5-HTP), cholecystokinin fragment (CCK), delta-sleep inducing peptide (DSIP), ephedra extract, and/or any other suitable substance. Accordingly, the stimulation signal applied to the user, including the signal delivery parameter value(s) associated with one or more of the cognates/substances, is expected to effectuate one or more mental and/or physical states in the user, including: calm, relaxed, alert, focused, happy, sleepy, deep sleep, tired, bedtime, boost, pain relief, and/or any other suitable mental and/or physical states. In at least some embodiments, for example, (i) a stimulation regiment that is meant to effectuate the same response as THC is expected to cause the user to be placed in a calm state, (ii) a stimulation regiment that is meant to effectuate the same response as CBD is expected to cause the user to be placed in a relaxed state, (iii) a stimulation regiment that is meant to effectuate the same response as caffeine is expected to cause the user to be placed in an alert state, (iv) a stimulation regiment that is meant to effectuate the same response as nicotine is expected to cause the user to be placed in a focused state, (v) a stimulation regiment that is meant to effectuate the same response as alcohol is expected to cause the user to be placed in a happy state, (vi) a stimulation regiment that is meant to effectuate the same response as melatonin is expected to cause the user to be placed in a sleepy or deep sleep state, (vii) a stimulation regiment that is meant to effectuate the same response as adenosine is expected to cause the user to be placed in a tired or bedtime state, and/or (viii) a stimulation regiment that is meant to effectuate the same response as theobromine is expected to cause the user to be placed in an energized or boost state. One or more of the stimulation regiments can cause/produce/induce biological and/or neurological activity associated with one or more of the mental states, sensations, feelings, etc., and/or biological and/or neurological activity corresponding with one or more of the substances associated with a given stimulation regiment.

A user can select one or more of the stimulation regiments based at least partially on the user's desired state (e.g., restful, energized, focused, pain relief, alert, happy, sleepy, etc.) and/or responsive to the user's current state. For each stimulation regiment, one or more of the associated signal delivery parameters may change over time based on a variety of factors, such as user-provided inputs, time of day, duration of the stimulation regiment, predetermined sequences including multiple stimulation regiments, real-time feedback based at least partially on information associated with the user (e.g., taken from sensors associated with the body support system 100 and/or other devices communicating therewith, such as smart watches). For example, at least some of the stimulation regiments may cause the user to feel calm or tired, e.g., to lull the person into sleep and/or a meditative state (e.g., in the evening), and/or to feel awake or energized (e.g., in the morning). In the illustrated embodiment, for example, the body support system 100 is positioned on a mattress (as described above), and individual ones of the emission elements 122 are configured to deliver a first stimulation regiment having one or more first signal delivery parameters at a first time (e.g., evening) expected to produce a calming state, e.g., to help the first user fall asleep faster and/or improve the first user's sleep quality, and can be further configured to emit a second stimulation regiment at a second time (e.g., morning) having one or more second signal delivery parameters expected to produce an energized state, e.g., to help the first user wake up and/or reduce drowsiness. The body support system 100 can transition between the first and second stimulation regiments based on, e.g., the time of day, a user's sleep schedule, a predetermined stimulation regiment sequence for improved restfulness/sleep quality, data associated with the user's sleeping habits or behaviors, etc.

In some embodiments, the emission assembly 110 can be configured to deliver multiple stimulation regiments concurrently or in sequence via one or more of the emission elements 122. In some embodiments, for example, during a given time period, one or more of the emission elements 122 can deliver a first stimulation regiment for a set period of time and a second stimulation regiment different than the first stimulation regiment for a subsequent period of time period, e.g., alternating between the first and second stimulation regiments. Additionally, or alternatively, one or more of the emission elements 122 can deliver a first stimulation regiment and a second stimulation regiment that have been combined or merged together, e.g., to form a single or joint stimulation regiment, such as by using digital signal processing techniques. In these and other embodiments, one or more of the emission elements 122 can deliver a first stimulation regiment and one or more other emission elements 122 can emit a second stimulation regiment different than the first stimulation regiment during the overlapping time periods to produce a combination of effects of the first and second stimulation regiments, thereby allowing the first and second stimulation regiments to be combined or “mixed” with each other. The user can adjust the mix of two or more differing stimulation regiments, the pattern in which they are applied, and/or the duration of application of each signal. In some embodiments, specific recipes of combined signals or patterns can be automated based on predefined recipes provided by the controller 114 and/or feedback from sensors that receive information from the user. In some embodiments, a plurality of emission elements 122 can be positioned in relatively close proximity (e.g., clustered together) such that each of the plurality of emission elements 122 (i) are positioned to stimulate a generally similar or identical region of the user's body (e.g., lower back) and (ii) can deliver a specific stimulation regiment, such that the region of the user's body region can receive a plurality of stimulation regiments, each from at least one of the plurality of emission elements 122.

The emission assembly 110 is operably coupled to the controller 114 and/or the control station 116. In the illustrated embodiment, for example, each of the emission elements 122 are operably coupled to the controller 114 and/or the control station 116 by one or more wires 124. In other embodiments, one or more of the emission elements 122 can be wirelessly coupled to the controller 114 and/or the control station 116 (via, e.g., Bluetooth, WiFi, or any other suitable wireless connection). Additionally, each of the emission elements 122 can be operably coupled to a power source 118 (e.g., a battery, a wall outlet, etc.) directly and/or via the controller 114 (as shown in FIG. 1) and/or the control station 116.

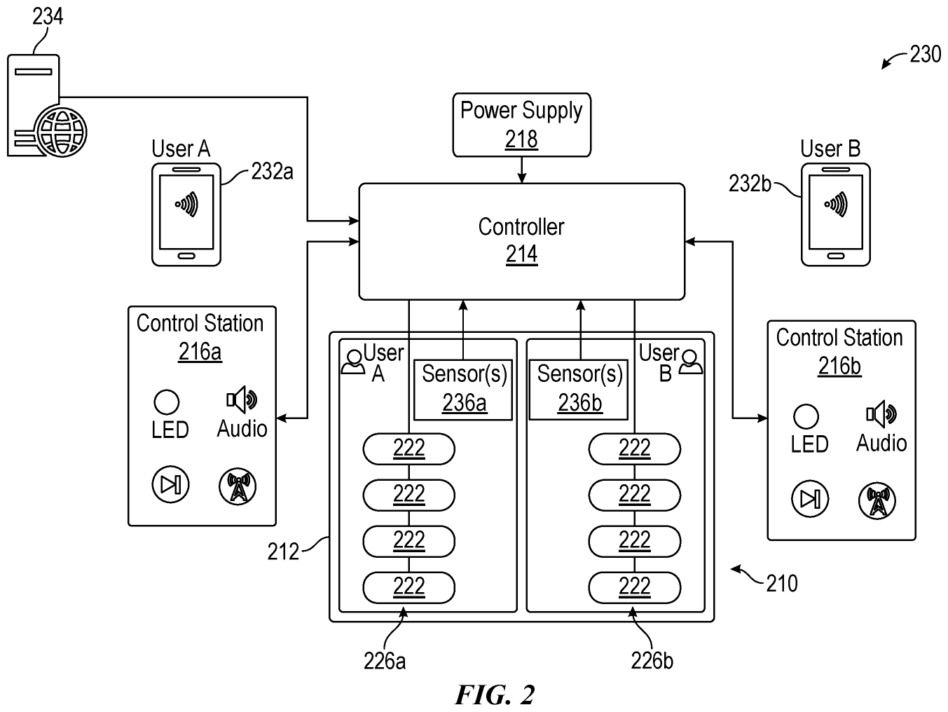

The controller 114 and/or the control station 116 can include or be communicatively couple to a user interface, such as buttons or a touch screen to allow a user to control the emission assembly 110. In some embodiments, the user interface may be on a separate device, such as an application running on a smart phone, tablet, or computer, as described below with reference to FIG. 2. The controller 114 can be configured to select and/or determine one or more signal delivery parameter values and cause the emission assembly 110 (e.g., individual ones of the emission elements 122) to deliver one or more stimulation regiments having the selected/determined signal delivery parameter values. Additionally, the controller 114 can change or update individual signal delivery parameter and/or signal delivery parameter values, e.g., in response to a user input, based on a predetermined pattern, etc., as described above. In these and other embodiments, the controller 114 can merge or combine two or more stimulation regiments, e.g., to form a joint stimulation regiment as described above. The control station 116 can be configured to selectively activate and/or deactivate the controller 114 and/or the emission assembly 110, e.g., to transition the emission assembly 110 and/or individual emission elements 122 between an “ON” state and an “OFF” state. Although in the illustrated embodiment the controller 114 is coupled (e.g., mechanically, communicably, operably, and/or the like) to the emission assembly 110 via a wired connection and the control station 116 is coupled to controller 114 via a wired connection, in other embodiments the controller 114, the control station 116, and/or the emission assembly 110 can be coupled via a wired or wireless connection (e.g, Bluetooth, WiFi, and the like), or via any other suitable connection. In at least some embodiments, the controller 114 and/or the control station 116 can integrated into the emission assembly 110.

FIG. 2 is a partially schematic illustration of an energy-emitting body support system 230 and associated environment configured in accordance with some embodiments of the present technology. Elements of the energy-emitting body support system 230 can be generally similar to or identical in structure and/or function as the features of the body support system 100 described above with respect to FIG. 1, with like numbers (e.g., emission assembly 210 versus the emission assembly 110 of FIG. 1) indicating generally similar or identical elements.

In the illustrated embodiment, the emission assembly 210 is configured for multi-user operation, e.g., to apply stimulation to a first user (e.g., User A, as shown in FIG. 2) and a second user (e.g., User B, as shown in FIG. 2). The first row 226a of emission elements 222 is operably coupled to a first control station 216a via the controller 214, and the second row 226b of emission elements 222 is operably coupled to a second control station 216b via the controller 214. Additionally, or alternatively, one or more of the emission elements 222 in the first row 226a can be operably coupled to a first electronic device 232a (e.g., a mobile phone, a tablet, etc.) associated with the first user and/or one or more of the emission elements 222 in the second row 226b can be operably coupled to a second electronic device 232b associated with the second user, such that the users of the first and second electronic devices 232a, 232b can enable, disable, or otherwise adjust the stimulation applied by individual ones of the emission elements 222 in the respective first and second rows 226a, 226b. In some embodiments, the first and/or second electronic devices 232a, 232b can be configured to communicate (e.g., transmit and/or receive information, data, signal delivery parameters, and the like) with the emission elements 222 via the respective first and second control stations 216a, 216b and/or via the controller 214 using a wired and/or a wireless communication link (e.g., LAN, NFC, Bluetooth, WiFi, and the like). In at least some embodiments, for example, an application (e.g., “the app,” “the mobile app,” “the web app,” and the like) running at least partially on the first and/or second electronic devices 232a, 232b can be used to select/change the signal delivery parameter values and/or stimulation regiments applied by individual ones of the emission elements 222 in the respective first and second rows 226a, 226b.

The controller 214 can support multiple communication channels (independent and/or overlapping) and/or connections with each of the emission elements 222, such that each of the emission elements 222 can emit a same or different signal and/or at least some of the emission elements 222 can emit a plurality of signals. Each of the control stations 216a, 216b can include an optical feedback component (e.g., an LED light), and audio component (e.g., a microphone, a speaker, etc.), a communication component (e.g., WiFi antenna, Bluetooth antenna, etc.), and a user input component (e.g., an ON/OFF button or switch). The user can activate/deactivate stimulation regiments by actuating the user input component. The optical feedback component can indicate whether the respective control station 216a, 216b and associated emission element(s) 222 are ON or OFF. The audio component can provide audio feedback associated with the operation of the emission assembly 210 and/or receive user voice commands. The communication component can pair the respective control stations 216a, 216b with the controller 214, one or both of the electronic devices 232a, 232b, the emission assembly 210, etc. Additionally, the controller 214, the first control station 216a, the second control station 216b, the first electronic device 232a, and/or the second electronic device 232b can connect to the internet and/or a remote computing device or server 234, e.g., to send and/or receive data for product software and/or firmware updates, emission assembly 210 operation, user data transfer and/or analysis, etc.

In some embodiments, the emission assembly 210 further includes one or more sensors 236 positioned to sense/detect data associated with one or more of the users. In the illustrated embodiment, for example, the emission assembly 210 includes a first sensor 236a positioned to sense first data associated with the first user and a second sensor 236b positioned to sense second data associated with the second user. In other embodiments, the emission assembly 210 include more or fewer sensors. The sensors 236a-b can include one or more motion sensors (e.g., an accelerometer), temperature sensors, sound sensors (e.g., a microphone), light sensors, cameras, force sensors (e.g, a pressure sensor, a weight sensor, and the like), one or more biometric sensors (e.g., a heart rate sensor, a blood oxygen sensor, a body temperature sensor, a weight sensor, and the like) and/or any other suitable sensors. Each of the sensors 236a-b can be communicatively connected to the controller 214, the respective control stations 216a-b, and/or the respective electronic devices 232a-b, such that the sensors 23 6a-b can transmit data associated with one or more of the users to the controller 214, the respective control stations 216a-b, and/or the respective electronic devices 232a-b. In some embodiments, individual ones of the first and/or second sensors 236a-b are embedded in, carried by, and/or otherwise coupled to the emission assembly substrate 212 (e.g., carried by or embedded in a mattress and/or mattress topper). In other embodiments, individual ones of the first and/or second sensors 236a-b are separate from the emission assembly substrate 212. In these and other embodiments, each of the sensors 236a-b can be communicatively coupled to the controller 214, the respective control station 216a-b, the respective electronic device 232a-b (e.g., the app), and/or with the remote computing device 234.

The data received from the sensors 236a-b can be used to determine various characteristics of the user and/or to adjust the operation of the emission assembly 210. For example, the first sensor 236a may detect the first user's sleep state and/or other physical measurements. This information can be communicated to the controller 214, one or more of the control stations 216a-b, one or more of the electronic devices 232a-b, and/or the remote computing device 234 for analysis and/or processing, e.g., to determine whether the signal being emitted by one or more of the emission elements 222 in the first row 226a is producing the desired effect in the first user (e.g., suitable for the sleep state) and/or change (e.g., modify, stop, enhance, or otherwise alter) one or more of associated signal delivery parameter values to improve or enhance the desired effect (e.g., improve the first user's sleep state and/or sleep quality). In the illustrated embodiment, the sensors 236a-b may be used to determine the respective user's 236a-b sleeping position (e.g., via pressure sensors) and/or position relative to one or more of the emission elements 222 and, based at least partially on the determined position of one or both of the users, change one or more of the signal delivery parameter values (e.g., activate a different emission element 222 closer to the user, increase the amplitude of the applied electrical stimulation, etc.).

The stimulation regiments applied to a user can be set or determined in a number of ways. In some embodiments, the user can program one or more signal delivery parameters, e.g., to program their own stimulation regiment or recipe. In some embodiments, the user can download one or more stimulation regiments, e.g., from a web site or database, such as the remote computing device 234. In some embodiments, the user can stream one or more stimulation regiments in real time, e.g., via the remote computing device 234. In some embodiments, one or more stimulation regiments can be automatically recommended and/or determined specifically for the user, e.g., based on the user's data obtained by the sensors 236a-b.

In some embodiments, the operation of the emission assembly 210 can be adjusted based on the number of users using the emission assembly 210. In the illustrated embodiment, for example, the emission assembly 210 has the first row 226a of emission elements 222 for the first user and the second row 226b of emission elements 222 for the second user, and the controller 214 can recognize the number of users using the emission assembly 210 (based on, e.g., the number of active/inactive control stations 216a-b, an input from one or more of the electronic devices 232a-b, an input from one or more of the sensors 236a-b, etc.) and, accordingly, automatically transition the emission assembly 210 between a single-user mode and a multi-user mode. In the single-user mode, the emission assembly 210 can only activate one of the rows 226a-b or may configure one row (e.g., the second row 226b) to mirror the operation of the other row (e.g., the first row 226a), such that both rows 226a-b apply the same stimulation regiment(s). In the multi-user mode, the emission assembly 210 can operate the first and second rows 226a, 226b independently of each other, as described above. Additionally, in the multi-user mode, one user's stimulation regiment(s) can be adjusted based at least partially on changes to another user's stimulation regiment(s). For example, if the controller 214 determines that a first user is sleeping fitfully (e.g., tossing and turning), the controller 214 may adjust the first user's stimulation regiment to address the first user's fitful sleep and also adjust a second user's stimulation regiment to reduce or prevent the first user's fitful sleep from impacting the second user's sleep.

Although in the illustrated embodiment the body support system 230 includes a single emission assembly 210, in other embodiments the body support system 230 can include multiple emission assemblies, each of which can be generally similar or identical in structure and/or function to the emission assembly 210. In such embodiments, a user's stimulation regiment(s) can be transferred between individual emission assemblies based at least partially on the user's location. In at least some embodiments, for example, the body support system 230 includes a chair emission assembly and a mattress emission assembly. The body support system 230 can detect when the user is seated in the chair emission assembly (based, e.g., on a sensor in the chair emission assembly, proximity of a user's electronic device to the chair emission assembly, a communication link between the user's electronic device and the chair emission assembly, a user input, etc.) and activate the chair emission assembly to deliver one or more stimulation regiments to the user. If the user gets out of the chair and moves to the mattress, the environment can detect the user's movement, deactivate the chair emission assembly, activate the mattress emission assembly, and deliver (e.g., resume delivery of) one or more stimulation regiments to the user.

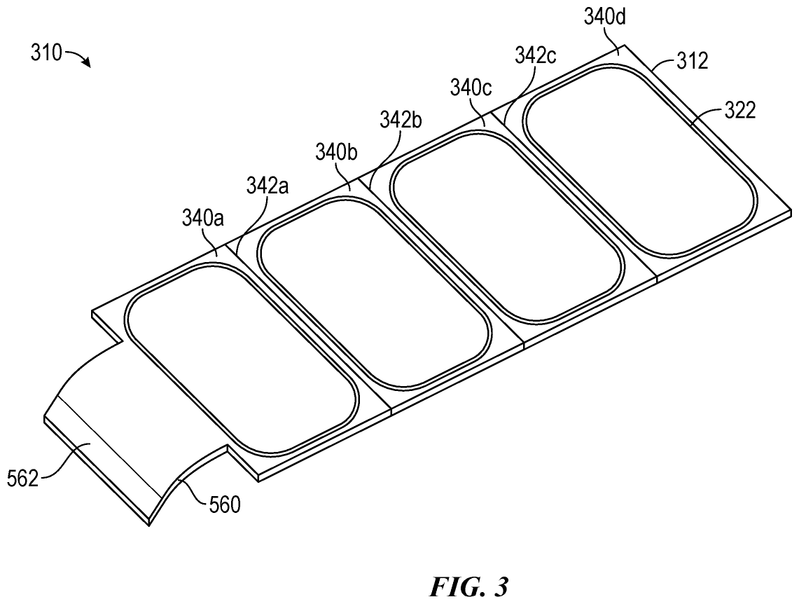

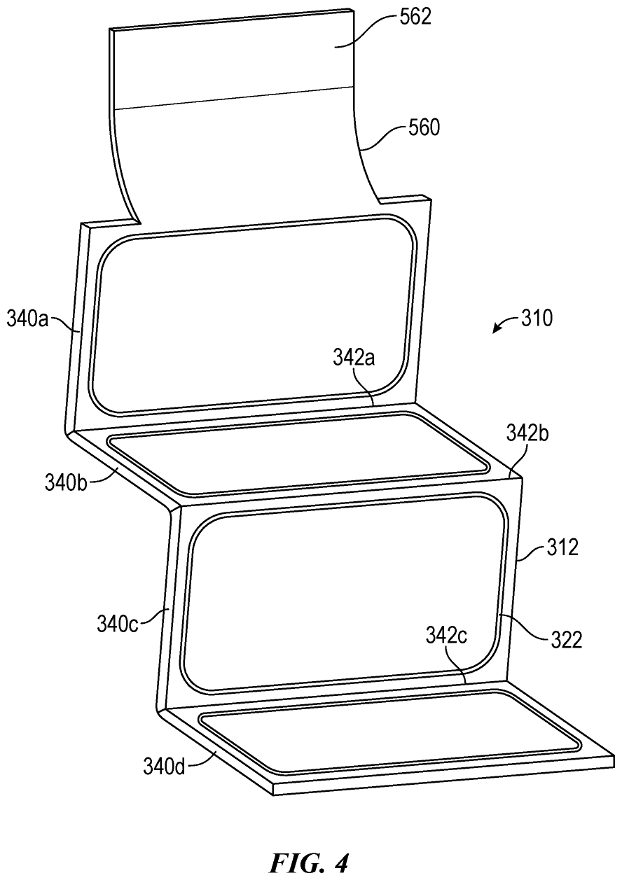

FIGS. 3-5 are isometric views of an emission assembly 310 in a flat state, a partially folded state, and a folded state, respectively, in accordance with embodiments of the present technology. The partially folded configuration shown in FIG. 4 is one example of a possible partially folded configuration of the emission assembly 310 provided to illustrate aspects of the present technology. In other embodiments, other partially folded configurations are possible, in addition to or in lieu of the partially folded configuration shown in FIG. 4.

Referring to FIGS. 3-5 together, the emission assembly 310 can be generally similar or identical in structure and/or function to the emission assembly 110 of FIG. 1 and/or the emission assembly 210 of FIG. 2, with like numbers (e.g., emission assembly substrate 312 versus the emission assembly substrate 112, 212 of FIGS. 1 and 2, respectively) indicating generally similar or identical elements. In some embodiments, the emission assembly 310 can be placed on a seated surface (e.g., a chair) and/or integrated into a seated surface (e.g., a chair seat, a chair back, car seat, bus seat, train seat, etc.) in the unfolded, the partially folded, and/or the folded configuration. In some embodiments, the emission assembly 310 can be placed beneath a user and at least partially aligned with one or more portions of the user's anatomy (e.g., back, arms, legs, head, etc.).

The emission assembly 310 includes a plurality of regions 340 (which can also be referred to as “the segments 340,” “the panels 340,” and the like). In the illustrated embodiment, the emission assembly 310 includes four regions 340a-d (e.g., a first region 340a, a second region 340b, a third region 340c, and a fourth region 340d) arranged in a line. In other embodiments, the emission assembly 310 can include any other suitable number of regions 340, such as less than four regions or more than four regions, and/or any other suitable arrangement of the regions, such as an L-shaped arrangement, a T-shaped arrangement, a V-shaped arrangement, a U-shaped arrangement, an X-shaped arrangement, a ring-shaped arrangement, etc.

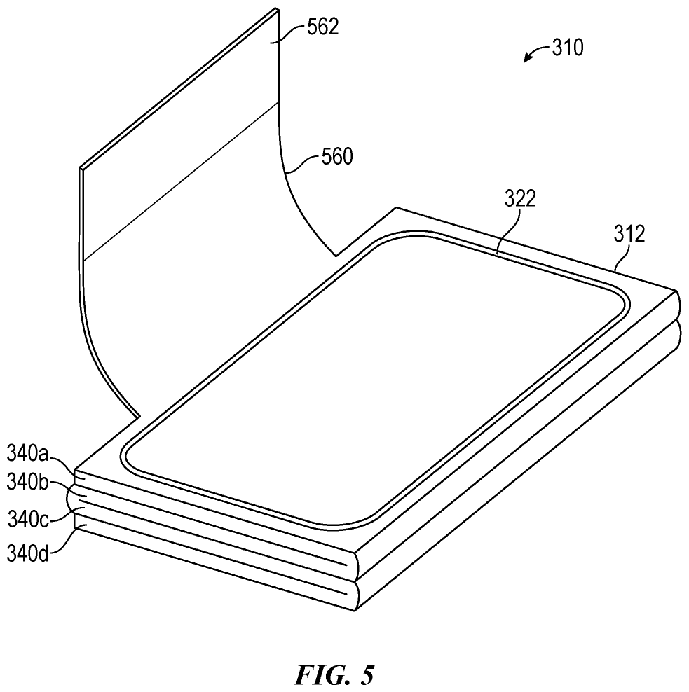

Individual ones of the regions 340 can be a portion of the emission assembly substrate 312 and/or a discrete component coupled to one or more other regions 340. Additionally, individual ones of the regions 340 can move or pivot relative to each other, e.g., about respective folding axes 342a-c positioned between individual ones of the regions 340 (e.g., as shown in FIG. 4). Each of the folding axis 342a-c can extend partially or fully across a dimension (e.g., a width or length) of the emission assembly 310. Each of the folding axes 342a-c can be a portion (e.g., a thinner portion, an indented portion, a perforated portion, etc.) of the emission assembly substrate 312 and/or include one or more flexible materials, hinges, and/or other connection structures configured such that individual ones of the regions (e.g., the first region 340a) can fold over onto a neighboring region (e.g., the second region 340b) to transition the emission assembly 310 between an unfolded configuration or state (e.g., FIG. 3), a partially folded configuration or state (e.g., FIG. 4), and a folded configuration or state (e.g., FIG. 5). In the folded configuration (FIG. 5), the regions 340 can be “stacked” or otherwise aligned such that the emission assembly 310 is more compact (e.g., reduced surface area) compared to the unfolded configuration (FIG. 3), e.g., to allow for improved storage and/or easier transport.

Each of the regions 340 can include one or more emission elements 322. In the illustrated embodiment, for example, each of the regions 340a-d includes one emission element 322. In other embodiments, individual ones of the regions 340a-d can include more or fewer emission elements 322, such as zero emission elements, more than one emission element, or any other suitable number of emission elements. When the emission assembly 310 is in the folded configuration (FIG. 5), one or more of the emission elements 322 in one region (e.g., the first region 340a) can be at least partially aligned with one or more other emission elements 332 in another region (e.g., the second region 340b, the third region 340c, and/or the fourth region 340d). In some embodiments, activating one or more of the emission elements 322 in multiple regions 340 when the emission assembly 310 is in the folded configuration (FIG. 5) can increase the overall stimulation output of the emission assembly 310, e.g., due at least partially to the aligned or “stacked” arrangement of the emission elements 322.

Referring to FIG. 5, in some embodiments the emission assembly 310 includes a strap or closure member 560 operable to maintain the emission assembly 310 in the folded configuration. In the illustrated embodiment, for example, the strap 560 is coupled to the first region 340a and is configured to releasably couple one or more of the other regions 340b-d. In some embodiments the strap 560 includes a coupling portion 562, and the coupling portion is configured to releasably couple one or more of the regions 340. The coupling portion 562 can include Velcro®, one or more adhesives, one or more magnets, and/or any other suitable coupling elements.

### EXAMPLES

Several aspects of the present technology are set forth in the following examples:

1. An energy-emitting body support system for a human body, the energy-emitting body support system comprising:
a controller having instructions to generate a stimulation regiment including an ultralow radiofrequency energy signal configured to produce a predefined physiological effect on the human body;
an emission assembly operably coupled to the controller, wherein the emission assembly comprises a plurality of emission elements sized and shaped to deliver the stimulation regiment to at least a portion of the human body when the human body is positioned thereon, and wherein the controller is configured to individually control each of the plurality of emission elements; and
an emission assembly substrate carrying the emission assembly, wherein the emission assembly substrate is configured to support at least the portion of the human body.

2. The energy-emitting body support system of example 1 wherein the emission assembly substrate comprises a flexible padding.

3. The energy-emitting body support system of example 1 or example 2 wherein the ultralow radiofrequency energy signal includes a frequency of less than 50 kHz and/or a magnetic field amplitude of up to about 50 mGauss.

4. The energy-emitting body support system of any of examples 1-3 wherein the emission assembly is configured to deliver the ultralow radiofrequency energy signal to produce a predefined effect on the human body, wherein the predefined effect includes at least one of a calm state, a tired state, a restful state, an energized state, and a motivated state, a relaxed state, an alert state, a focused state, a state of happiness, and/or a state of pain relief.

5. The energy-emitting body support system of example 4 wherein the stimulation regiment is configured to induce biological activity associated with the predefined effect.

6. The energy-emitting body support system of any of examples 1-5 wherein the plurality of emission elements comprises a first emission element sized and shaped to deliver the stimulation regiment to at least a first portion of the human body and a second emission element sized and shaped to deliver the stimulation regiment to at least a second portion of the human body.

7. The energy-emitting body support system of example 6 wherein the second portion of the human body is different than the first portion of the human body.

8. The energy-emitting body support system of example 6 or example 7 wherein the stimulation regiment is a first stimulation regiment, wherein the controller has instructions to
generate a second stimulation regiment,
deliver the first stimulation regiment via the first emission element, and
deliver the second stimulation regiment via the second emission element.

9. The energy-emitting body support system of example 8 wherein the first stimulation regiment differs from the second stimulation regiment.

10. The energy-emitting body support system of example 8 or example 9 wherein the predefined physiological effect is a first predefined physiological effect, wherein the first stimulation regiment is configured to produce the first predefined physiological effect on the human body, and wherein the second stimulation regiment is configured to produce a second predefined physiological effect on the human body.

11. The energy-emitting body support system of example 10 wherein the second predefined physiological effect is different than the first predefined physiological effect.

12. The energy-emitting body support system of any of examples 8-11 wherein the first stimulation regiment includes a signal delivery parameter having a first signal delivery parameter value, wherein the second stimulation regiment includes the signal delivery parameter having a second signal delivery parameter value, and wherein the first signal delivery parametervalue differs from the second signal delivery parameter value.

13. The energy-emitting body support system of any of examples 1-12 wherein at least one of the plurality of emission elements includes a coil.

14. The energy-emitting body support system of any of examples 1-13 wherein at least one of the plurality of emission elements includes an electronic layer.

15. An energy-emitting body support system for a human body, the energy-emitting body support system comprising:
a support substrate configured to support at least a portion of the human body,
an emission assembly coupled to the support substrate, wherein
   the emission assembly includes one or more emission elements, and
   individual ones of the one or more emission elements are sized and shaped to deliver a stimulation regiment to at least part of the portion of the human body when the human body is positioned thereon;
a controller operably coupled to the emission assembly and having instructions to generate the stimulation regiment, wherein the stimulation regiment includes an ultralow radiofrequency energy signal having one or more signal delivery parameter values and configured to produce a predefined physiological effect on the human body; and
a sensor communicably coupled to the controller and positioned to detect data associated with the human body;
wherein the controller is configured to adjust at least one of the one or more signal delivery parameter values based at least partially on the data detected by the sensor.

16. The energy-emitting body support system of example 15 wherein the sensor includes at least one of a motion sensor, a temperature sensor, a sound sensor, a light sensor, a camera, a pressure sensor, a weight sensor, a heart rate sensor, a blood oxygen sensor, and/or a body temperature sensor.

17. The energy-emitting body support system of example 15 or example 16 wherein the emission assembly is operably coupled to an electronic device, and wherein the electronic device is communicably coupled to an app configured to adjust at least one of the one or more signal delivery parameter values.

18. The energy-emittingbody support system of example 17 wherein the electronic device includes a mobile phone.

19. The energy-emitting body support system of any of examples 15-18 wherein the emission assembly is a first emission assembly and the sensor is a first sensor positioned to detect first data, the energy-emitting body support system further comprising:
a second emission assembly; and
a second sensor positioned to detect second data;
wherein the controller is configured to transfer the stimulation regiment between the first emission assembly and the second emission assembly based at least partially on the first data and the second data.

20. A method of producing a predefined physiological effect on a human body with an energy-emitting body support system, the method comprising:
receiving, via the energy-emitting body support system, an input associated with a stimulation regiment, wherein the stimulation regiment includes an ultralow radiofrequency energy signal and is configured to produce the predefined physiological effect; and
in response to the input, delivering, via at least one emission element of the energy-emitting body support system, the stimulation regiment to the human body when the human body is positioned on the energy-emitting body support system, wherein delivering the stimulation regiment includes producing the predefined physiological effect.

21. The method of example 20, further comprising:
receiving, from a sensor of the energy-emitting body support system, data associated with the human body; and
adjusting at least one signal delivery parameter value of the stimulation regiment based at least partially on the received data.

22. The method of example 20 or example 21 wherein receiving the input includes receiving the input via an electronic device operably coupled to the at least one emission element.

23. The method of example 22 wherein the electronic device includes a mobile phone.

24. The method of any of examples 20-23, wherein the at least one emission element is at least one first emission element, the method further comprising:
receiving, from a sensor of the energy-emitting body support system, data associated with a changed position of the human body; and
based at least partially on the received data
   stopping delivery of the stimulation regiment via the at least one first emission element, and/or
   delivering the stimulation regiment via at least one second emission element of the energy-emitting body support system.

### CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, the use of “or” in such a list is to be interpreted as including (i) any single item in the list, (ii) all of the items in the list, or (iii) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. An energy-emitting body support system for a human body, the energy-emitting body support system comprising:
a controller having instructions to generate a stimulation regiment including an ultralow radiofrequency energy signal configured to produce a predefined physiological effect on the human body;
an emission assembly operably coupled to the controller, wherein the emission assembly comprises a plurality of emission elements sized and shaped to deliver the stimulation regiment to at least a portion of the human body when the human body is positioned thereon, and wherein the controller is configured to individually control each of the plurality of emission elements; and
an emission assembly substrate carrying the emission assembly, wherein the emission assembly substrate is configured to support at least the portion of the human body.

2. The energy-emitting body support system of claim 1 wherein the emission assembly substrate comprises a flexible padding.

3. The energy-emitting body support system of claim 1 wherein the ultralow radiofrequency energy signal includes a frequency of less than 50 kHz and/or a magnetic field amplitude of up to about 50 mGauss.

4. The energy-emitting body support system of claim 1 wherein the emission assembly is configured to deliver the ultralow radiofrequency energy signal to produce a predefined effect on the human body, wherein the predefined effect includes at least one of a calm state, a tired state, a restful state, an energized state, and a motivated state, a relaxed state, an alert state, a focused state, a state of happiness, and/or a state of pain relief.

5. The energy-emitting body support system of claim 4 wherein the stimulation regiment is configured to induce biological activity associated with the predefined effect.

6. The energy-emitting body support system of claim 1 wherein the plurality of emission elements comprises a first emission element sized and shaped to deliver the stimulation regiment to at least a first portion of the human body and a second emission element sized and shaped to deliver the stimulation regiment to at least a second portion of the human body.

7. The energy-emitting body support system of claim 6 wherein the second portion of the human body is different than the first portion of the human body.

8. The energy-emitting body support system of claim 6 wherein the stimulation regiment is a first stimulation regiment, wherein the controller has instructions to
generate a second stimulation regiment,
deliver the first stimulation regiment via the first emission element, and
deliver the second stimulation regiment via the second emission element.

9. The energy-emitting body support system of claim 8 wherein the first stimulation regiment differs from the second stimulation regiment.

10. The energy-emitting body support system of claim 8 wherein the predefined physiological effect is a first predefined physiological effect, wherein the first stimulation regiment is configured to produce the first predefined physiological effect on the human body, and wherein the second stimulation regiment is configured to produce a second predefined physiological effect on the human body.

11. The energy-emitting body support system of claim 10 wherein the second predefined physiological effect is different than the first predefined physiological effect.

12. The energy-emitting body support system of claim 8 wherein the first stimulation regiment includes a signal delivery parameter having a first signal delivery parameter value, wherein the second stimulation regiment includes the signal delivery parameter having a second signal delivery parameter value, and wherein the first signal delivery parameter value differs from the second signal delivery parameter value.

13. The energy-emitting body support system of claim 1 wherein at least one of the plurality of emission elements includes a coil.

14. The energy-emitting body support system of claim 1 wherein at least one of the plurality of emission elements includes an electronic layer.

15. An energy-emitting body support system for a human body, the energy-emitting body support system comprising:
a support substrate configured to support at least a portion of the human body,
an emission assembly coupled to the support substrate, wherein
the emission assembly includes one or more emission elements, and
individual ones of the one or more emission elements are sized and shaped to deliver a stimulation regiment to at least part of the portion of the human body when the human body is positioned thereon;
a controller operably coupled to the emission assembly and having instructions to generate the stimulation regiment, wherein the stimulation regiment includes an ultralow radiofrequency energy signal having one or more signal delivery parameter values and configured to produce a predefined physiological effect on the human body; and
a sensor communicably coupled to the controller and positioned to detect data associated with the human body;
wherein the controller is configured to adjust at least one of the one or more signal delivery parameter values based at least partially on the data detected by the sensor.

16. The energy-emitting body support system of claim 15 wherein the sensor includes at least one of a motion sensor, a temperature sensor, a sound sensor, a light sensor, a camera, a pressure sensor, a weight sensor, a heart rate sensor, a blood oxygen sensor, and/or a body temperature sensor.

17. The energy-emitting body support system of claim 15 wherein the emission assembly is operably coupled to an electronic device, and wherein the electronic device is communicably coupled to an app configured to adjust at least one of the one or more signal delivery parameter values.

18. The energy-emitting body support system of claim 17 wherein the electronic device includes a mobile phone.

19. The energy-emitting body support system of claim 15 wherein the emission assembly is a first emission assembly and the sensor is a first sensor positioned to detect first data, the energy-emitting body support system further comprising:
a second emission assembly; and
a second sensor positioned to detect second data;
wherein the controller is configured to transfer the stimulation regiment between the first emission assembly and the second emission assembly based at least partially on the first data and the second data.

20. A method of producing a predefined physiological effect in a human body with an energy-emitting body support system, the method comprising:
receiving, via the energy-emitting body support system, an input associated with a stimulation regiment, wherein the stimulation regiment includes an ultralow radiofrequency energy signal and is configured to produce the predefined physiological effect; and
in response to the input, delivering, via at least one emission element of the energy-emitting body support system, the stimulation regiment to the human body when the human body is positioned on the energy-emitting body support system, wherein delivering the stimulation regiment includes producing the predefined physiological effect.

21. The method of claim 20, further comprising:
receiving, from a sensor of the energy-emitting body support system, data associated with the human body; and
adjusting at least one signal delivery parameter value of the stimulation regiment based at least partially on the received data.

22. The method of claim 20 wherein receiving the input includes receiving the input via an electronic device operably coupled to the at least one emission element.

23. The method of claim 22 wherein the electronic device includes a mobile phone.

24. The method of claim 20, wherein the at least one emission element is at least one first emission element, the method further comprising:
receiving, from a sensor of the energy-emitting body support system, data associated with a changed position of the human body; and
based at least partially on the received data
stopping delivery of the stimulation regiment via the at least one first emission element, and/or
delivering the stimulation regiment via at least one second emission element of the energy-emitting body support system.
